(19) United States
(12) Patent Application Publication
(10) Pub. No.: US 2013/0150831 A1
(43) Pub. Date: Jun. 13, 2013

(54) SYSTEM AND METHOD FOR AN ARTICULATING DISTAL END OF AN ENDOSCOPIC MEDICAL DEVICE

(71) Applicant: Jerry R. GRIFFITHS, Pembroke, MA (US)

(72) Inventor: Jerry R. GRIFFITHS, Pembroke, MA (US)

(73) Assignee: SYMMETRY MEDICAL NEW BEDFORD, INC, New Bedford, MA (US)

(21) Appl. No.: 13/711,844

(22) Filed: Dec. 12, 2012

Related U.S. Application Data
(60) Provisional application No. 61/570,122, filed on Dec. 13, 2011.

(51) Int. Cl. A61B 17/00

(52) U.S. Cl. CPC A61B 17/00
USPC 606/1

(57) ABSTRACT

An endoscopic medical device system includes an elongated shaft assembly and an articulating distal end assembly. The elongated shaft assembly extends along a main axis and includes an outer tube, an inner tube, and an actuator rod. The outer tube is slidably disposed around the inner tube and the actuator rod is slidably disposed within the inner tube. The articulating distal end assembly extends from the inner tube and is configured to be oriented longitudinally along the main axis when disposed within the outer tube and angularly from the main axis as it slides out of the outer tube. The articulating distal end assembly includes a tubular body having equally spaced apart slots separated by solid segments and terminates in a distal segment. Each slot is configured to collapse individually and to form a known angle relative to the main axis as it slides out of the outer tube.

### CROSS REFERENCE TO RELATED CO-PENDING APPLICATIONS

This application claims the benefit of U.S. provisional application Ser. No. 61/570,122 filed on Dec. 13, 2011 and entitled SYSTEM AND METHOD FOR AN ARTICULATING DISTAL END OF AN ENDOSCOPIC MEDICAL DEVICE which is commonly assigned and the contents of which are expressly incorporated herein by reference.

### FIELD OF THE INVENTION

The present invention relates to a system and a method for an articulating distal end of an endoscopic medical device, and in particular, to an articulating distal end that moves angularly relative to the device's main axis

### BACKGROUND OF THE INVENTION

Endoscopic medical instruments with articulating distal ends are used in various surgical procedures for off-axis drilling, inserting implants in a specific orientation, accessing and grasping organs in a specific direction, and suturing. Prior art systems usually include distal ends that articulate via a cable or an actuator rod system. In most of the prior art systems the articulating distal ends are not held rigid in their angular position. This may result in uncontrolled and imprecise drilling, movement of the implant, abrasion of the tissue or organ being grasped, injury of the surrounding tissue or ineffective suturing.

Accordingly there is a need for improved methods and systems that allow articulation of a distal end of an endoscopic medical instrument while holding the distal end in a rigid position.

### SUMMARY OF THE INVENTION

The present invention provides an endoscopic medical device having an elongated shaft with an articulating distal end that moves angularly from the main shaft axis. The articulating distal end is used to change the orientation of an end-effector device and to hold the end-effector device in a rigid position.

In general, in one aspect, the invention features an endoscopic medical device system including an elongated shaft assembly and an articulating distal end assembly. The elongated shaft assembly extends along a main axis and includes an outer tube, an inner tube, and an actuator rod. The outer tube is slidably disposed around the inner tube and the actuator rod is slidably disposed within the inner tube. The articulating distal end assembly extends from the inner tube and is configured to be oriented longitudinally along the main axis when disposed within the outer tube and angularly from the main axis as it slides out of the outer tube. The articulating distal end assembly includes a tubular body having equally spaced apart slots separated by solid segments and terminates in a distal segment. Each slot is configured to collapse individually and to form a known angle relative to the main axis as it slides out of the outer tube.

Implementations of this aspect of the invention may include one or more of the following features. The actuator rod includes a flexible elongated body and a bent distal end and the bent distal end is configured to engage an opening formed in the distal segment of the articulating distal end assembly. The outer tube is configured to move backwards along the main axis simultaneously with the actuator rod while maintaining a constant spatial ratio relative to the actuator rod. Each slot has an open end and a closed end and each slot is configured to sequentially slide out of the outer tube as the outer tube moves backwards along the main axis, and the simultaneous backward motion of the actuator rod flexes inward the distal segment and sequentially closes the open end of each exposed slot, thereby causing each slot to collapse individually. The value of the known angle depends upon the tension applied on the actuator rod and the portion of the tubular body that slides out of the outer tube. The system further includes a handle assembly and the handle assembly includes a handle, an upper body connected to the top of the handle, and a rotation knob. The elongated shaft assembly extends from the upper body and the outer tube and the actuator rod are configured to move simultaneously longitudinally backwards or forwards along the main axis, via counter-clockwise or clockwise rotation of the rotation knob. The articulating distal end assembly further includes an end effector and the end effector may be one of a drill guide, a drill bit, scissors, grasping jaws, cutting blades or shavers. The system may further include an implantable component configured to be delivered and implanted through the inner tube. The inner tube and outer tube comprise one of stainless steel, titanium, gold, silver, Nitinol or alloys thereof and the actuator rod comprises one of stainless steel, titanium, Nitinol or alloys thereof

In general, in one aspect, the invention features a method for controlling the angular orientation of an articulating distal end assembly including the following. Providing an endoscopic medical device comprising an elongated shaft assembly, and an articulating distal end assembly. The elongated shaft assembly extends along a main axis and includes an outer tube, an inner tube, and an actuator rod, and the outer tube is slidably disposed around the inner tube and the actuator rod is slidably disposed within the inner tube. The articulating distal end assembly extends from the inner tube and is configured to be oriented longitudinally along the main axis when disposed within the outer tube and angularly from the main axis as it slides out of the outer tube. The articulating distal end assembly includes a tubular body having equally spaced apart slots separated by solid segments and terminates in a distal segment. The method further includes collapsing each slot individually and thereby orienting the articulating distal end assembly at a known angle relative to the main axis as it slides out of the outer tube.

Among the advantages of this invention may be one or more of the following. The articulating distal end provides controlled angular movement of the end-effector device relative to the main shaft axis, while maintaining the distal end in a rigid position. Through the entire range of motion the distal end remains very stiff and is not springy. The present design includes a hollow inner tube that functions as conduit for inserting a drill bit or medical implants or devices to the surgical site via minimally invasive surgery (MIS). The articulating distal end is formed as a single component having multiple slots. This reduces the manufacturing cost and the complexity of the system relative to current designs that include multiple articulating links. The details of one or more embodiments of the invention are set forth in the accompanying drawings and description below. Other features, objects and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1 is a side perspective view of an endoscopic medical device with an articulating distal end according to this invention;

FIG. 2A and FIG. 2B are exploded views of the shaft of the device of FIG. 1;

FIG. 3A is a side view of the shaft of the device of FIG. 1 depicting the distal end in the articulated orientation;

FIG. 3B is a side view of the shaft of the device of FIG. 1 depicting the distal end in a partially articulated orientation;

FIG. 3C is a side view of the shaft of the device of FIG. 1 depicting the distal end in the straight orientation;

FIG. 4 is a detailed view of the articulating distal end of the device in FIG. 3A in the articulated orientation;

FIG. 5 is a detailed view of the articulating distal end of the device in FIG. 3B in the partially articulated orientation;

FIG. 6 is a detailed view of the articulating distal end with a drill bit end effector; and

FIG. 7 is a detailed view of the articulating distal end with a tissue grasper end effector.

### DETAILED DESCRIPTION OF THE INVENTION

The present invention provides an endoscopic medical device having an elongated shaft with an articulating distal end that moves angularly from the main shaft axis. The articulating distal end is used to change the orientation of an end-effector device and to hold the end-effector device in a rigid position.

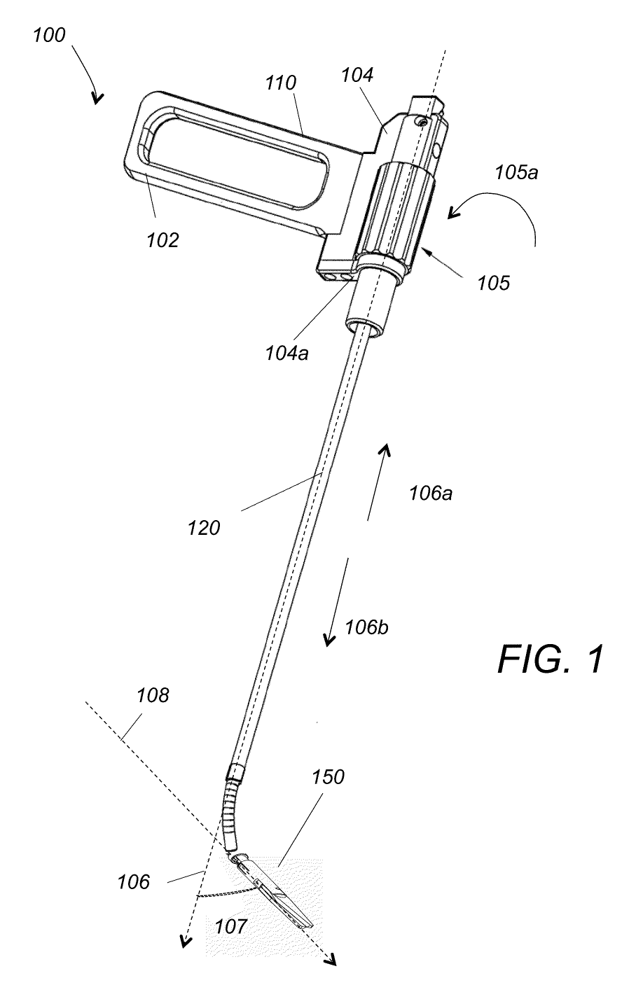

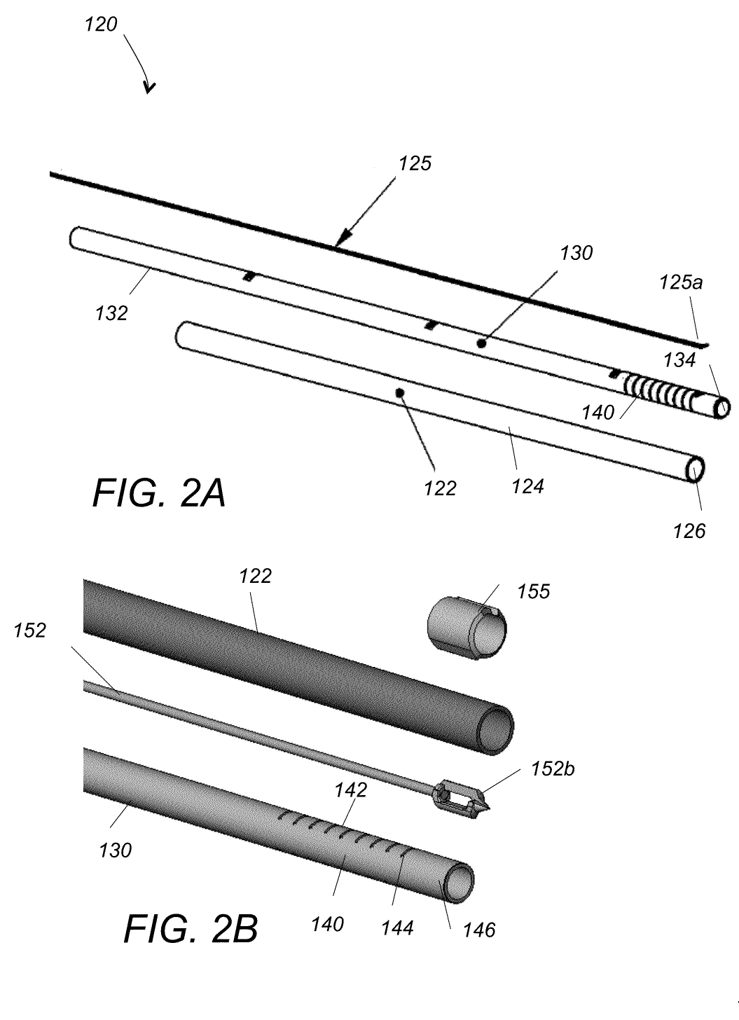

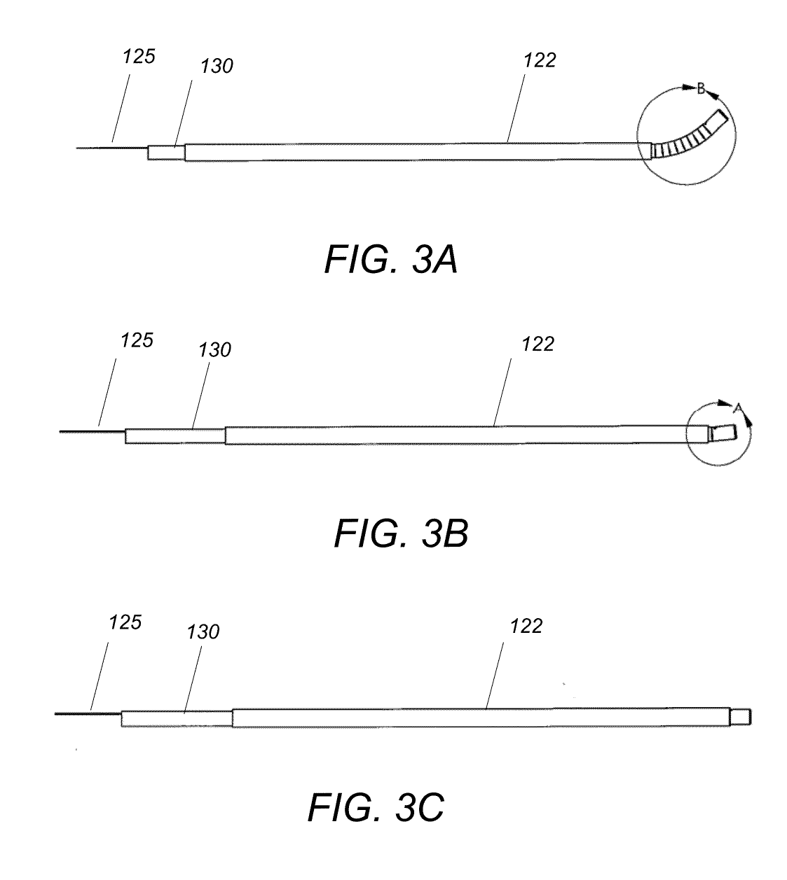

Referring to FIG. 1, endoscopic medical device 100 includes a handle assembly 110, a shaft assembly 120 and a distal end assembly 150. Handle assembly 110 includes a handle 102, an upper body 104 and a rotation knob 105. Shaft assembly 120 extends from end 104a of the upper body 104 along axis 106 and includes an outer tube 122, an inner tube 130, a flexible actuator rod 125, and an outer drill point 155, as shown in FIG. 2A and FIG. 2B. Inner tube 130 is positioned within outer tube 122 and flexible actuator rod 125 is positioned within inner tube 130. Outer tube 122 has a solid cylindrical main body 124 extending along axis 106 and forms a lumen 126. Outer tube 122 is slip fit over inner tube 130 and is configured to translate axially over inner tube 130 along axis 106 via rotation of rotation of knob 105. Inner tube 130 includes a solid cylindrical body 132 and has an articulating distal end 140. Cylindrical body 132 forms a lumen 134 within which actuator rod 125 moves axially along axis 106. Distal end 140 is configured to articulate and move from a substantially linear configuration (shown in FIG. 3C) to a substantially angled, off-axis or articulated configuration (shown in FIG. 3A). In the articulated configuration, distal end 140 is bent along axis 108 and forms an angle 107 with axis 106, as shown in FIG. 1. Flexible actuator rod 125 includes a flexible elongated body and a bent distal end 125a that is configured to engage an opening or a notch 148 formed in the distal end 140, as shown in FIG. 2A and will be described below. Actuator rod 125 is configured to move axially along direction 106 via the rotation of knob 105 simultaneously with the axial movement of the outer tube 122 along direction 106.

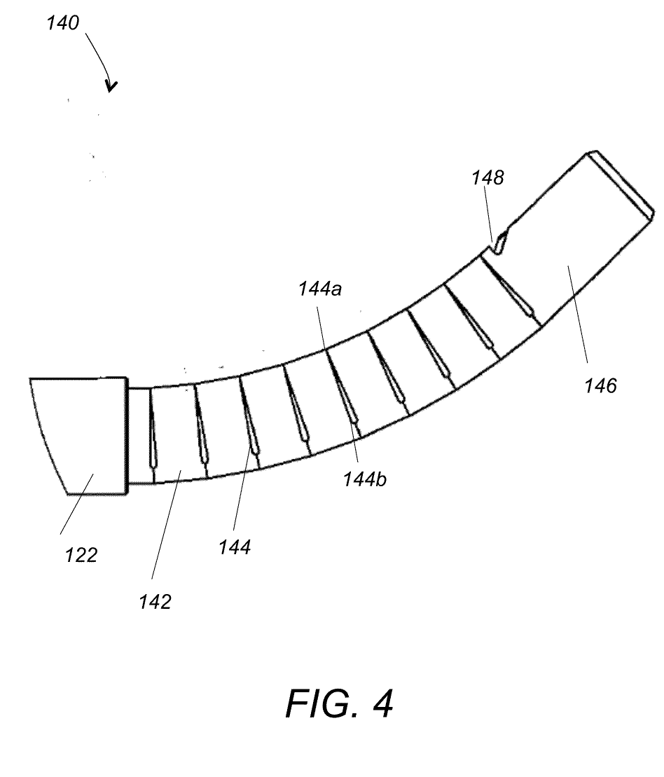

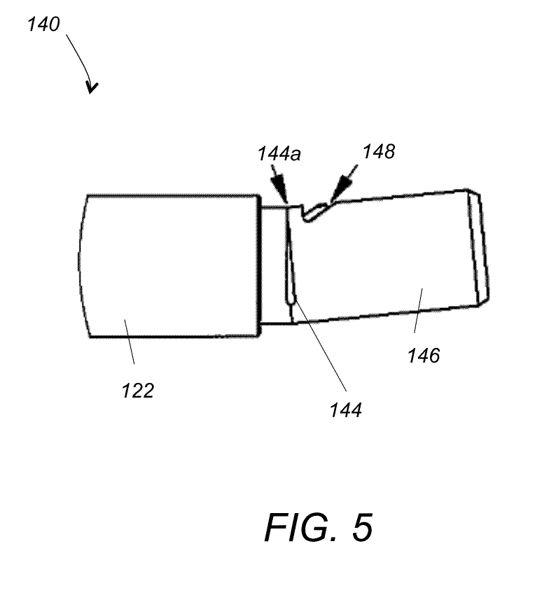

Referring to FIG. 2A, FIG. 2B and FIG. 4, distal end 140 includes equally spaced apart slots 144 separated by solid segments 142 and terminates in distal segment 146. Slots 144 extend perpendicular to axis 106 and have an open end 144a and a closed end 144b, as shown in FIG. 4. Open end 144a is pinched closed when the distal end 140 is flexed in order to assume the articulated configuration, shown in FIG. 3A. Distal segment 146 includes a notch 148 that is configured to engage the distal end 125a of the flexible actuator rod 125.

In operation, turning the rotation knob 105 counter-clockwise 105a retracts the outer tube along direction 106a and pulls the flexible actuator rod 125 simultaneously along direction 106a. In one example, the outer tube 122 moves back relative to the flexible actuator rod 125 at a ratio of 8:1. This backward motion of the outer tube 122 exposes the slotted distal end 140 of the inner tube 130, while the simultaneous backward motion of the flexible actuator rod 125 flexes the distal end 140 and closes the open ends 144a of slots 144. As the open ends 144a of slots 144 are flexed together, slots 144 collapse and bent the distal end 140 along axis 108, which is set at an angle 107 from the longitudinal device axis 106. The measure of angle 107 depends upon the tension applied upon the flexible actuator rod 125 and the portion of the distal end 140 that is exposed as the outer tube is retracted. As shown in FIG. 3A, the distal end 140 is fully tensed and exposed (articulated configuration). In this configuration each slot is fully collapsed and intension and the distal end 140 of the inner shaft 130 remains rigid. In FIG. 3B, the distal end is partially tensed and exposed (partially articulated configuration). Reversing the rotation of knob 105 (i.e., clockwise rotation) reverses the actuation mechanism and causes the actuator rod 125 and the outer tube 122 to move simultaneously forward along direction 106b. The forward motion of the outer tube 122 relaxes the distal end 140 and opens the slots 144 thereby causing the distal end 140 to straighten and to become collinear with the longitudinal device axis 106. Simultaneously, the forward motion of the outer tube 125 translates the outer tube 125 over the straightened slotted distal end 140.

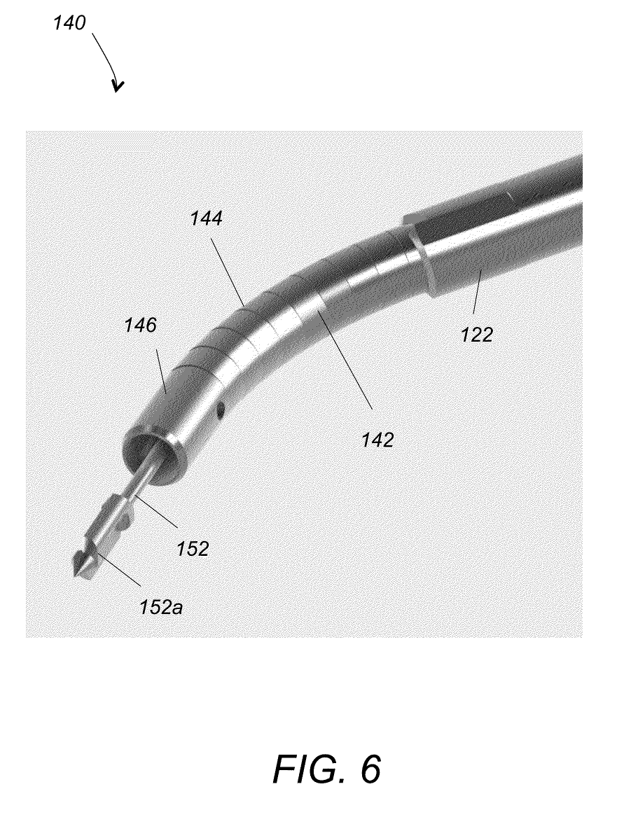

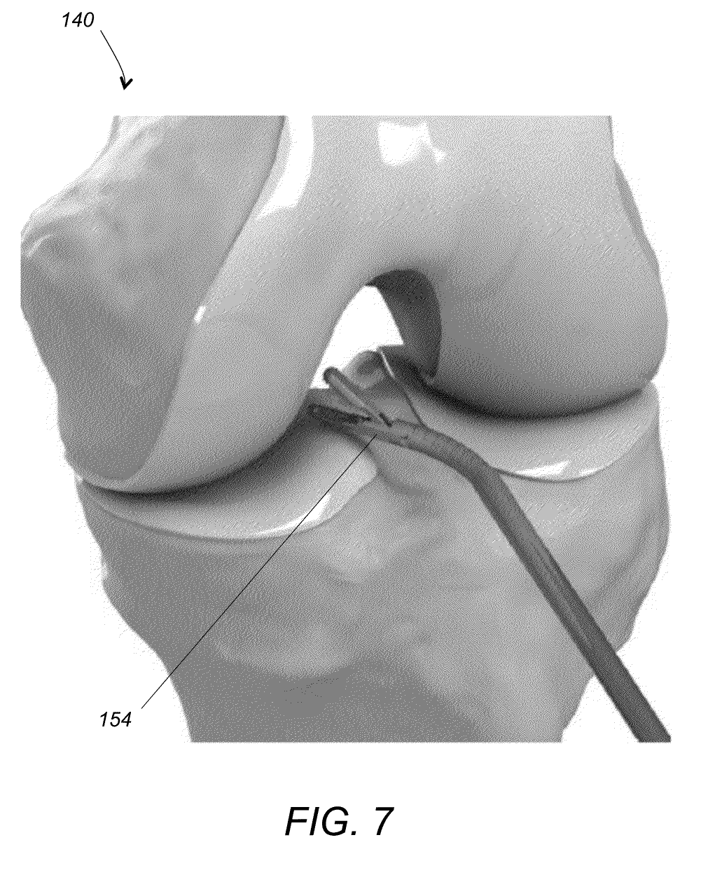

Distal end assemblies 150 that attach to the articulating distal ends may be drill guides, drill bits 152a (shown in FIG. 6), scissors, grasping jaws 154 (shown in FIG. 7), cutting blades, spade drills 152b (shown in FIG. 2B), drill guides, and shavers, among others. Since the articulated distal end is held in a rigid position it is suitable for precise off-axis drilling and for holding and manipulating precisely an implant during laparoscopic or other endoscopic surgical procedures. The hollow inner tube 130 may also be used to deliver surgical or implant components through its center lumen 134 to the surgical site via minimally invasive surgery(MIS).

Inner tube 130 and outer tube 122 are manufactured from a variety of materials including among others stainless steel, titanium, gold, silver Nitinol, or alloys thereof Flexible actuator rod 125 is manufactured or materials including Nitinol, stainless steel, or titanium, among others.

Several embodiments of the present invention have been described. Nevertheless, it will be understood that various modifications may be made without departing from the spirit and scope of the invention. Accordingly, other embodiments are within the scope of the following claims.

What is claimed is:

1. An endoscopic medical device system comprising:
an elongated shaft assembly extending along a main axis and comprising an outer tube, an inner tube, and an actuator rod, and wherein the outer tube is slidably disposed around the inner tube and the actuator rod is slidably disposed within the inner tube;
an articulating distal end assembly extending from the inner tube and configured to be oriented longitudinally along the main axis when disposed within the outer tube and angularly from the main axis as it slides out of the outer tube; and
wherein the articulating distal end assembly comprises a tubular body having equally spaced apart slots separated by solid segments and terminates in a distal segment and wherein each slot is configured to collapse individually and to form a known angle relative to the main axis as it slides out of the outer tube.

2. The system of claim 1, wherein the actuator rod comprises a flexible elongated body and a bent distal end and wherein the bent distal end is configured to engage an opening formed in the distal segment of the articulating distal end assembly.

3. The system of claim 2, wherein the outer tube is configured to move backwards along the main axis simultaneously with the actuator rod while maintaining a constant spatial ratio relative to the actuator rod.

4. The system of claim 3, wherein each slot comprises an open end and a closed end and wherein each slot is configured to sequentially slide out of the outer tube as the outer tube moves backwards along the main axis, and wherein the simultaneous backward motion of the actuator rod flexes inward the distal segment and sequentially closes the open end of each exposed slot, thereby causing each slot to collapse individually.

5. The system of claim 4, wherein the value of the known angle depends upon the tension applied on the actuator rod and the portion of the tubular body that slides out of the outer tube.

6. The system of claim 1, further comprising a handle assembly and wherein the handle assembly comprises a handle, an upper body connected to the top of the handle, and a rotation knob.

7. The system of claim 6, wherein the elongated shaft assembly extends from the upper body and the outer tube and the actuator rod are configured to move simultaneously longitudinally backwards or forwards along the main axis, via counter-clockwise or clockwise rotation of the rotation knob.

8. The system of claim 1, wherein the articulating distal end assembly further comprises an end effector and wherein the end effector comprises one of a drill guide, a drill bit, scissors, grasping jaws, cutting blades or shavers.

9. The system of claim 1 further comprising an implantable component configured to be delivered and implanted through the inner tube.

10. The system of claim 1 wherein the inner tube and outer tube comprise one of stainless steel, titanium, gold, silver, Nitinol or alloys thereof and wherein the actuator rod comprises one of stainless steel, titanium, Nitinol or alloys thereof

11. A method for controlling the angular orientation of an articulating distal end assembly comprising:
providing an endoscopic medical device comprising an elongated shaft assembly, and an articulating distal end assembly;
wherein the elongated shaft assembly extends along a main axis and comprises an outer tube, an inner tube, and an actuator rod, and wherein the outer tube is slidably disposed around the inner tube and the actuator rod is slidably disposed within the inner tube;
wherein the articulating distal end assembly extends from the inner tube and is configured to be oriented longitudinally along the main axis when disposed within the outer tube and angularly from the main axis as it slides out of the outer tube; and
wherein the articulating distal end assembly comprises a tubular body having equally spaced apart slots separated by solid segments and terminates in a distal segment; and
collapsing each slot individually and thereby orienting the articulating distal end assembly at a known angle relative to the main axis as it slides out of the outer tube.

12. The method of claim 11, wherein the actuator rod comprises a flexible elongated body and a bent distal end and wherein the bent distal end is configured to engage an opening formed in the distal segment of the articulating distal end assembly.

13. The method of claim 12, wherein the outer tube is configured to move backwards along the main axis simultaneously with the actuator rod while maintaining a constant spatial ratio relative to the actuator rod.

14. The method of claim 13, wherein each slot comprises an open end and a closed end and wherein each slot is configured to sequentially slide out of the outer tube as the outer tube moves backwards along the main axis, and wherein the simultaneous backward motion of the actuator rod flexes inward the distal segment and sequentially closes the open end of each exposed slot, thereby causing each slot to collapse individually.

15. The method of claim 14, wherein the value of the known angle depends upon the tension applied on the actuator rod and the portion of the tubular body that slides out of the outer tube.

16. The method of claim 11, wherein the endoscopic medical device further comprises a handle assembly and wherein the handle assembly comprises a handle, an upper body connected to the top of the handle, and a rotation knob.

17. The method of claim 16, wherein the elongated shaft assembly extends from the upper body and the outer tube and the actuator rod are configured to move simultaneously longitudinally backwards or forwards along the main axis, via counter-clockwise or clockwise rotation of the rotation knob.

18. The method of claim 11, wherein the articulating distal end assembly further comprises an end effector and wherein the end effector comprises one of a drill guide, a drill bit, scissors, grasping jaws, cutting blades or shavers.

19. The method of claim 11, further comprising providing an implantable component and delivering and implanting the implantable component through the inner tube.

20. The method of claim 11, wherein the inner tube and outer tube comprise one of stainless steel, titanium, gold, silver, Nitinol or alloys thereof and wherein the actuator rod comprises one of stainless steel, titanium, Nitinol or alloys thereof.
